(19) United States
(12) Patent Application Publication
(10) Pub. No.: US 2010/0036177 A1
(43) Pub. Date: Feb. 11, 2010

(54) PROCESSING OF NATURAL OIL-BASED PRODUCTS HAVING INCREASED VISCOSITY

(76) Inventor: Derik J. Ward, Apple Valley, MN (US)

Correspondence Address: CARGILL, INCORPORATED, P.O. Box 5624, MINNEAPOLIS, MN, 55440-5624, US

(21) Appl. No.: 12/515,169

(22) PCT Filed: Nov. 16, 2007
(86) PCT No.: PCT/US07/24153

§ 371 Date: Oct. 12, 2009

Related U.S. Application Data
(60) Provisional application No. 60/859,448, filed on Nov. 16, 2006.

(51) Int. Cl. C07C 31/18

(52) U.S. Cl. 568/865

(57) ABSTRACT

Methods for the removal of volatilizable components from a natural oil-based product having a high viscosity are provided. The natural oil-based product can be a high viscosity oligomeric polyol prepared from epoxidized vegetable oils. The method involves heating a natural oil-based product to relatively low temperatures (about 225° C. or below) and then exposing the natural oil-based product to an environment comprising a reduced pressure and a sparging vapor. In particular, deodorization methods are provided. In deodorization processes, odor components such as hexanal, nonanal, and decanal are substantially removed from the natural oil-based product. The lower temperature process provides effective removal of the volatilizable components, while maintaining desirable product properties, such as the clarity of the product, and chemical and physical properties similar to that of the starting material.

### CROSS REFERENCE TO RELATED APPLICATION

This application claims the benefit of PCT Patent Application Serial No. PCT/US2007/024153 filed Nov. 16, 2007 entitled PROCESSING OF NATURAL OIL-BASED PRODUCTS HAVING INCREASED VISCOSITY, which claims the benefit of U.S. Provisional Patent Application Ser. No. 60/859,448 filed Nov. 16, 2006 entitled PROCESSING OF NATURAL OIL-BASED PRODUCTS HAVING INCREASED VISCOSITY, which are hereby incorporated by reference in their entirety.

### FIELD

The invention relates to methods for removing volatilizable compounds from natural oil-based products having elevated viscosities. In particular aspects, the invention relates to deodorized natural oil-based products and methods for deodorizing these products.

### BACKGROUND

Polyols produced from vegetable oil-based products have more recently been prepared as an alternative to polyols produced from petroleum. As compared to petroleum based polyols, vegetable oil-based polyols have the advantage of being renewable resources.

Polyols produced from vegetable oil-based products can be used in a variety of applications, such as for coatings, adhesives, sealants, elastomers, resins and foams. Polyols may be used in a wide variety of fields including the textile, plastic, medical, chemical, manufacturing, and cosmetic industries. Some particular uses of vegetable oil-based polyols include the production of flexible polyurethane foams, which are typically made by the reaction of polyols or polyol compositions with organic polyisocyanates. Flexible polyurethane foams have various commercial and industrial applications including cushioning, load-bearing, and comfort-providing components of bedding and transportation articles.

The production of vegetable oil-based polyols is known in the art (some examples of non-petroleum based polyols include those described by Petrovic et al. in U.S. Pat. Nos. 6,107,433, 6,433,121, 6,573,354, and 6,686,435). Polyol production from vegetable oil is exemplified by the ring opening reaction of epoxidized vegetable oil. As a first step, the epoxidation of a vegetable oil is carried out using an oxidizing compound such as peracetic acid. The polyol is created by reacting the epoxidized oil with a nucleophile in the presence of an acid catalyst, typically fluoroboric acid (HBF₄). Depending on the particular method used, a polyol product can be formed having a desired saturation level and desired hydroxyl functionality.

In many cases, polyol formation results in a significant increase in viscosity of the product. The actual increase in viscosity of the product may depend on various properties of the polyol product, such as the saturation levels, hydroxyl functionality and relative oligomerization of the polyol product.

Despite advantages that vegetable oil-based starting materials can provide for the production of polyols, there are some issues associated with starting materials used to form polyols, and/or process of polyol synthesis that can negatively impact the polyol product, and potentially any product formed therefrom.

In particular, one issue relates to the presence and generation of odor components. Odor components that are present in vegetable oils typically include volatilizable (low boiling point) compounds such as short chain (C₁₄ and less) aldehydes, alcohols, carboxylic acids, ketones, and esters. In some cases, polyols prepared from blown soybean oil (such as described in Kurth, U.S. Pat. No. 6,180,686) inherently possess high concentrations of these volatile, highly odorous compounds.

Vegetable oils typically include odor components that are subsequently reduced or removed in steps associated with the production of refined, bleached and deodorized (RBD) oils. Deodorization of vegetable oils, such as soybean oil, is carried out at very high temperatures of above 250° C. and under very low vacuum (1 to 6 mm Hg). Although deodorization processes can reduce odor components to low levels, odor levels in commercially available RBD oils are known to vary greatly depending on the manufacturer and type of oil used.

Odor components present in vegetable oil starting materials can be carried over to the polyol synthesis process and remain in the polyol product. This can affect polyol quality and potentially any product formed therefrom. For example, odor components can be carried over into the polyol, which can subsequently be reacted to form a polyurethane product, such as a polyurethane foam used in a consumer product. This is undesirable as it can impart an unpleasant odor to the consumer product, and may negatively affect consumer reaction.

In addition, odor components can be generated during polyol production. In particular, unwanted reactions following epoxidation of the vegetable oil can lead to the production of various odor components along with the polyol product. Chain scission of epoxidized vegetable oil can lead to the generation of aliphatic aldehydes that can impart distinct odors to the polyol product C₆-C₄ aliphatic aldehydes such as hexanal, nonanal, and decanal can be generated in polyol production. Again, these odors can be undesirably carried over into products (such as polyurethane foams) produced from the polyol product.

The presence of peroxides and hydroperoxides in a polyol product can also cause production of odor components. Peroxides and hydroperoxides can be formed by the reaction of oxygen with unsaturated fatty acids in polyol precursors. Present in the polyol product, the peroxides and hydroperoxides can decompose. As an end product, hydrocarbons, aldehydes, and ketones can be produced, some of which can be odor components.

In addition, production of polyurethane foam may involve heating steps that are performed at high temperatures. These steps in the foaming process may result in the production of odor components if the polyol product as a starting material has not been adequately deodorized.

Furthermore, there are also concerns regarding application of high temperatures to the polyol product (such as would be required based on traditional deodorization processes). At these higher temperatures, unwanted changes in the polyol could take place. For example, thermal breakdown of the polyol can occur, leading to loss of product properties. In addition, use of high temperatures can cause the introduction of undesirable colors in the product. This can impart an undesirable color in a secondary product, such as a polyurethane foam.

### SUMMARY

The invention relates to processes for the removal of volatilizable components from natural oil-based products. The invention also relates to natural oil-based products having low levels of volatilizable components. The methods of the invention involve the use of natural oil-based products that have a viscosity higher than common vegetable oils (such as soybean oil). In particular, the invention concerns the removal of volatilizable components from natural oil-based products that have a viscosity of about 500 cP or greater at 25° C.

In more specific aspects, the invention relates to processes for the deodorization of natural oil-based products. The invention also relates to deodorized natural oil-based products. Natural oil-based products having this higher viscosity are exemplified by natural oil-based polyols, such as those derived from plant oils. Such natural oil-based polyols include polyols as well as oligomerized polyols. The polyol or oligomeric polyol can be made from an epoxidized plant oil, such as epoxidized soybean oil or epoxidized palm-based oil.

In some aspects the natural oil-based product is a polyol having a hydroxyl value of about 100 mg KOH/g or greater. For example, the polyol can have a hydroxyl value in the range of about 160-190 mg KOH/g, or a hydroxyl value in the range of about 200-250 mg KOH/g.

In other aspects, the natural oil-based product is an oligomeric polyol having a hydroxyl number of about 45 to about 65 mg KOH/g. In some cases the oligomeric polyol has a number average hydroxyl functionality (Fn) of less than about 2.7. In some cases the oligomeric polyol has about 40% weight or greater oligomers.

These polyol products have a viscosity that is significantly higher than the viscosity of the natural oils that are used to prepare the polyols. The increase is typically at least ten times the viscosity of the starting oils or greater, and more typically about twenty times the viscosity of the starting oils. In some specific aspects, the natural oil-based product has a viscosity of about 1000 cP or greater, 1500 cP or greater, 2000 cP or greater, 2500 cP or greater, or even 3000 cP or greater at 25° C. In some specific aspects the natural oil-based product has a viscosity of up to about 10000 cP, up to about 15000 cP, or up to about 20000 at 25° C.

In some aspects the natural oil-based product is a polyol having a particularly high viscosity. For example, the polyol can have a viscosity in the range of about 8000 cP to about 11000 cP at 25° C. As another example, the polyol can have a viscosity in the range of about 4000 cP to about 6000 cP at 25° C.

In some aspects the natural oil-based product is an oligomeric polyol having a high viscosity. For example, the oligomeric polyol can have a viscosity in the range of about 2500 cP to about 5000 cP at 25° C.

In some aspects, the volatilizable components removed from the natural oil-based products in the process of the present invention are odor components. These odor components may be present in the natural oil-based product as a result of being carried over from the starting material (e.g., vegetable oil), may be generated during production of the natural oil-based product, or both.

In other aspects, the volatilizable components removed from the natural oil-based products are components such as water or methanol. Removal of one or more of these components from the natural oil-based product can provide subsequent product and processing advantages. For example, removal of methanol can increase the flash point of a polyol product, allowing the polyol to be subsequently reacted in a broader range of conditions. Also, methanol may be detrimental in polyurethane production since it can act as a chain-terminating (capping) agent possibly affecting desirable properties of the polyurethane by limiting molecular weight.

Surprisingly, based on experimentation associated with the present invention it was found that removal of odor components from high viscosity polyol compositions was adequately performed (under reduced pressure and using sparging vapors) at temperatures lower than temperatures traditionally used for the deodorization of common vegetable oils. Prior to the inventive discovery, it was originally thought that a higher temperature would be required to facilitate processing of the natural oil-based product in the deodorization equipment given the higher viscosity of the product.

Despite this initial perception, test performed over a broad range of processing temperatures revealed that lower processing temperatures resulted in a substantial reduction of odor components. At the same time, these lower temperatures provided additional processing benefits, allowing the production of a natural oil-based polyol product with desirable properties.

Using these lower deodorization temperatures, desirable color characteristics associated with the natural oil-based product were advantageously maintained. In using the lower deodorization temperatures, thermal breakdown of the polyol product and undesirable yellowing were largely avoided. Following deodorization there was no increase, or at most an insignificant increase, in the color of the natural oil-based product. In some aspects, the deodorized natural oil-based products produced according to the methods of the invention have a color on the Gardner scale of about 2.0 or less, of about 1.5 or less, or about 1.0 or less. Therefore, the methods of the invention advantageously provide a deodorized natural oil based polyol having very low levels of odor components, as well as good color characteristics.

Despite use of the lower deodorization temperatures, the methods of the invention also provided a polyol product with low peroxide values. The lower peroxide values were a result of the effectiveness in reducing odor components during the deodorization process.

The methods of the invention are also beneficial from an economic standpoint. In one regard, the lower temperature deodorization steps necessitated less energy to be provided to the deodorization equipment. This can be important in scaled-up processes where industrial quantities of natural oil-based products are subjected to deodorization. Furthermore, since the methods of the invention involve temperatures that are generally lower than temperatures used in typical deodorization processes, the need for certain types of cooling equipment is not required.

In one aspect, the invention provides a process for removing volatilizable components from a natural oil-based product. In the process, a natural oil-based product is provided, the product having a viscosity of 500 cP or greater at 25° C. The process includes a step of heating the natural oil-based product to a temperature of about 225° C. or below. Following the step of heating, the process includes a step of exposing the natural oil-based product to an environment comprising a reduced pressure and a sparging vapor. The step of exposing is carried out for a period of time sufficient for removal of the majority of one or more volatilizable components that are associated with the natural oil-based product.

The heating step is performed at a temperature and for a period of time sufficient to reduce peroxide values in the natural oil-based product. As a result of the heating step, the peroxide values can be reduced by about 50% or greater, 60% or greater, 70% or greater, 80% or greater, or even 90% or greater. Reduction of peroxide value in the polyol product dramatically reduces the likelihood that products such as low boiling point odor components will be generated in a subsequent use or product of the natural oil-based product.

Preferably, in the step of heating, the natural oil-based product is heated to a temperature of about 220° C. or below. For example, in some modes of practice the product is heated to a temperature in the range of about 185° C. to about 220° C., or about 185° C. to about 218° C.; about 190° C. to about 220° C., or about 190° C. to about 218° C.; about 195° C. to about 220° C., or about 195° C. to about 218° C.; about 200° C. to about 220° C., or about 200° C. to about 218° C.; about 205° C. to about 220° C., or about 205° C. to about 218° C.; about 210° C. to about 220° C., or about 210° C. to about 218° C.; or most preferably, about 215° C. to about 220° C., or about 215° C. to about 218° C. Given this, the method of the present invention avoids heating to very high temperatures (above 225° C., such as 250° C.) for a period of time (such high temperatures are commonly used for the treatment of soybean oil).

In some preferred aspects, the step of heating comprises maintaining the natural oil-based product at the temperature of about 225° C. or below, such as at a temperature in a temperature range described herein, for a period of time in the range of about 8 min to about 15 minutes.

In another preferred aspect, in the step of exposing the natural oil-based product to an environment comprising a reduced pressure and a sparging vapor, the reduced pressure is maintained at about 5 Torr or below, and preferably at about 3 Torr or below. In another preferred aspect, in the step of exposing, the sparging vapor is provided in the range of about 0.5% (w/w) to about 2.0% (w/w), and more preferably in the range of about 1.0% (w/w) to about 2.0% (w/w) (weight of sparging vapor/weight of natural oil-based product).

In another preferred aspect, the step of exposing the natural oil-based product comprises flowing the natural oil-based product in a continuous countercurrent sparging vapor column. For example, after the natural oil-based product is heated according to the methods of the invention, it can be introduced into the column and can flow over packing materials. Due to the low vapor pressure in the column and the significant increase in the surface area between the natural oil-based product and the low pressure atmosphere in the column, the volatilizable components (e.g., odor components) are stripped from the product and are carried away from the product by a current of sparging vapors generated by the vacuum system.

In one aspect, the process is directed to removing odor components from the natural oil-based product. The process of the invention can be used to reduce the amount of at least one odor component associated with the natural oil-based product by an amount of 50% or greater. More typically the one or more odor component is reduced by an amount of about 60% or greater, 70% or greater, 80% or greater, 90% or greater, or (most typically) 95% or greater. In some aspects, the odor component is a saturated aldehyde. The saturated aldehyde can be selected from the group consisting of hexanal, nonanal and decanal.

In another aspect, the invention provides a natural oil-based product having reduced levels of volatilizable components. More particularly, the invention provides a deodorized natural oil-based product. The natural oil-based product has a viscosity of 500 cP or greater at 25° C.

In some specific aspects the deodorized natural oil-based product (e.g, a natural oil-based polyol) has a combined hexanal, nonanal, and decanal content of about 25 ppm or less. In some specific aspects the combined hexanal, nonanal, and decanal content is about 20 ppm or less, about 15 ppm or less, about 10 ppm or less, about 5 ppm or less, about 4 ppm or less, about 3 ppm or less, about 2 ppm or less, or about 1 ppm or less.

In some specific aspects, the invention provides a deodorized natural oil-based polyol having a combined hexanal, nonanal, and decanal content of about 25 ppm or less, and a color on the Gardner scale of about 2.0 or less. In some specific aspects, the invention provides a deodorized natural oil-based polyol having a combined hexanal, nonanal, and decanal content of about 5 ppm or less, and a color on the Gardner scale of about 1.5 or less.

For example, a process for deodorizing a natural oil-based polyol according to the invention can include the steps of (a) providing a natural oil-based polyol having a viscosity of 500 cP or greater (such as in the range of about 3000 cP to of about 11000 cP) at 25° C.; (b) heating the natural oil-based polyol to a temperature in the range of 185° C. to 220° C., wherein the temperature of the natural oil-based polyol in process does not exceed 220° C.; and (c) exposing the natural oil-based product to an environment comprising a reduced pressure and a sparging vapor, to provide a deodorized natural oil-based product having a combined hexanal, nonanal, and decanal content of 25 ppm or less and a Gardner color value of about 2 or less.

### BRIEF DESCRIPTION OF THE DRAWINGS

FIG. 1 is a graph showing total odor content (combined hexanal, nonanal, and decanal (ppm)) of the polyol samples following various deodorization runs, as described in Table 1.

FIG. 2 is a graph showing total odor content (combined hexanal, nonanal, and decanal (ppm)) of the polyol samples according to the polyol feed temperature in various deodorization runs.

FIG. 3 is a graph showing total odor content (combined hexanal, nonanal, and decanal (ppm)) of the polyol samples according to the retention time of the polyol (prior to introduction in the countercurrent sparging column) in various deodorization runs.

FIG. 4 is a graph showing total odor content (combined hexanal, nonanal, and decanal (ppm)) of the polyol samples according to the steam sparging rate in various deodorization runs.

FIG. 5 is a graph showing total odor content (combined hexanal, nonanal, and decanal (ppm)) of the polyol samples according to the pressure in the sparging column in various deodorization runs.

### DETAILED DESCRIPTION

In one aspect, the present invention provides methods for the removal of volatilizable components from a natural oil-based product having an elevated viscosity. In more specific aspects, the removal of volatilizable components results in the deodorization of the natural oil-based product.

The term “natural oil-based product” refers to natural oils, which include plant-based oils (e.g., vegetable oils) and animal fats that have been altered in a way that increases their viscosity. The alteration can be a modification of the natural oils by chemical reaction. The alteration of the natural oil results in a product of the natural oil having a viscosity that is substantially higher that that of the starting material (i.e., the natural oil). For example, a natural oil-based product with an “elevated viscosity” can have a viscosity that is about five time or greater, or ten times or greater than the viscosity measured of the natural oil starting material. The natural oil-based product with an elevated viscosity can be a polyol or an oligomeric polyol.

Viscosity is commonly measured in units Poise (P) or centipoise (cP), or Pascal seconds (pa·s.) using equipment such as a rotating spindle instrument, such as a Brookfield viscometer (Brookfield Engineering Laboratories, Middleboro, Mass.). The amount of force that is needed to turn the spindle (torque) is recorded in Poise (P) or centipoise (cP) (1.0 P=0.1 Newton-seconds/m²). The glass capillary viscometer is the standard instrument for measuring viscosity of Newtonian fluids and is calibrated with reference to the defined value of the viscosity of water.

Generally, it is known that various factors can affect the viscosity of a liquid composition. These include temperature and the purity of the composition (for example, wherein the presence of any one or more additional component(s) that may increase or reduce the viscosity of the liquid composition). For the natural oil-based products which can be subjected to the methods of the invention, viscosity of natural oil-based products can be determined when the product is at: (a) 95% (w/v) or greater, and (b) a temperature of 25° C.

According to the invention, a natural oil-based product with an “elevated viscosity” has a viscosity of about 500 cP (0.5 Pa s⁻¹) at 25° C. Natural oil-based products having a viscosity of greater than about 500 cP at 25° C. can also be effectively deodorized according to the methods of the present invention. For example, in more specific aspects, the natural oil-based product has a viscosity of about 1000 cP or greater, 1500 cP or greater, 2000 cP or greater, 2500 cP or greater, or even 3000 cP or greater at 25° C.

In some aspects the natural oil-based product, such as a polyol, has a viscosity in the range of about 1000 cP to about 20000 cP at 25° C., about 1000 cP to about 15000 cP at 25° C., or about 1000 cP to about 10000 cP at 25° C.

In some aspects the natural oil-based product, such as a polyol, has a viscosity in the range of about 3000 cP to about 20000 cP at 25° C., about 3000 cP to about 15000 cP at 25° C., or about 3000 cP to about 10000 cP at 25° C.

In some specific aspects the natural oil-based product, such as a polyol, has a viscosity in the range of about 2500 cP to about 5000 cP at 25° C.

In some specific aspects the natural oil-based product, such as a polyol, has a viscosity in the range of about 8000 cP to about 11000 cP at 25° C.

In some specific aspects the natural oil-based product, such as a polyol, has a viscosity in the range of about 4000 cP to about 6000 cP at 25° C.

As compared to the natural oil starting materials, the increase in viscosity of the natural oil-based product may be due to one or more of the following factors: an increase in the molecular weight of one or more components of the natural oil-based product composition; the addition of hydroxy groups onto the fatty acid chains; an increase in the ability of the one or more components of the natural oil-based product composition to exhibit hydrogen bonding; and/or an increase in the saturation of the one or more components of the natural oil-based product.

Another property of the natural oil-based product is its color. It is known that color properties of the natural oil-based product can be affected by processing conditions and/or chemical reactions. In many cases, it is desirable to provide a natural oil-based product that is deodorized but yet remains colorless, or that has low levels of color, rather than developing a yellow color.

Using methods of the present invention, which involve processing the natural oils based product at temperatures of about 225° C. or less, or about 220° C. or less, removal of volatilizable components can be accomplished without substantially increasing the coloration of the natural oil-based product. That is, the process can be carried out without heating the natural oil-based product to a temperature of greater than 225° C., or to a temperature of greater than 220° C., during the steps of the process, which may otherwise cause discoloration of the product.

“Volatilizable compounds” refers to low molecular weight compounds that can be present in, and removable from the natural oil-based product using heat, vacuum, or a combination of both. Volatilizable compounds include those having high, medium, low, and even very low volatilities. The methods of the present invention allow the significant reduction in the amount of volatilizable compounds in the natural oil-based product. The volatilizable compounds that can be removed can have from very low to high volatilities. However, it is recognized that compounds include those having high or medium volatilities may be more easily stripped from the natural oil-based product, and may be significantly reduced prior to the steps of heating and exposing. For example, before the step of exposing the polyol to a high vacuum and sparging vapor, the process may include a step of deaerating which is performed at about 100° C., and which can significantly reduce the amount of methanol in the natural oil-based product. The removal of any residual compound having a high volatility can take place in the step of exposing the polyol to a high vacuum and sparging vapor.

However, a significant reduction in the removal of compounds having low and very low volatilities generally does not occur until the steps of heating (less than 225° C.) and exposing.

An example of a compound having high volatility (for example, having a vapor pressure of greater than about 100 mmHg @ 20-25° C.) is methanol, which has a vapor pressure of 128 mmHg at 20° C. An example of a compound having a medium volatility (for example, having a vapor pressure in the range of about 25 to about 100 mmHg @ 20-25° C.) is butanal, which has a vapor pressure of 90 mm Hg (@ 20° C.). An example of a compound having a low volatility (for example, having a vapor pressure in the range of about 1 to about 25 mmHg @ 20-25° C.) is hexanal which has a vapor pressure of 10 mm Hg (@ 20° C.). Examples of a compounds having very low volatilities (for example, having a vapor pressure of less than about 1 mmHg @ 20-25° C.) are nonanal which has a vapor pressure of ˜0.26 mm Hg (@ 25° C.), and are decanal which has a vapor pressure of ˜0.15 mm Hg (@ 20° C.).

Color of the natural oil-based product can be determined using any suitable method known in the art. Color can be determined by visual inspection or by spectrophotometric analysis. For example, visual inspection of the natural oil-based product relative to the Gardner color scale can be performed. According to the Gardner color scale, the sample is evaluated and assigned a value in the range of 0-18 units, which provides the relative “yellowness” of the natural oil-based product. For example, an increase in coloration of the natural oil-based product is typically observed by an increase in intensity of yellow hues, and reflected by an increase in Gardner color scale units. Measurements according to ASTM D 1544 (visual Gardner color standard), ASTM D 6166 (instrumental determination of Gardner color), or AOCS Td 1A can be made.

For example, the ASTM D 6166 Standard Test Method, which uses an instrument such as a spectrophotometric calorimeter, can allow quantitative determination of the Gardner color of a natural oil-based product.

Color determination can be performed before, during, and/or after steps in the deodorization process. The test can provide values for the color of liquids by means of comparison with numbered glass standards. The methods of the present invention can be used so that the increase in color on the Gardner scale is not greater than 1 unit.

For example, in some aspects, prior to deodorization, the natural oil-based product can have a Gardner color value of less than 1 unit, and the deodorization process of the present invention can be carried out to provide a deodorized product having the about the same Gardner value, or a deodorized product having a Gardner color value of about 1 or less.

In some aspects, prior to deodorization the natural oil-based product can have a Gardner color value of about 1 unit, and the deodorization process of the present invention can be carried out to provide a deodorized product having a Gardner value of about 1, or about 1.5 or less.

In some aspects, prior to deodorization the natural oil-based product can have a Gardner color value of about 1.5 unit or less, and the deodorization process of the present invention can be carried out to provide a deodorized product having a Gardner value of about 2.0 or less.

Spectrophotometric analysis of samples before, during, and/or after steps in the deodorization process also be performed utilizing commercially available spectrophotometers.

In some aspects, the natural oil-based product with elevated viscosity is a natural oil that is reacted to provide additional oxygen-containing groups. For example, the natural oil-based product can be a polyol. As used herein “polyol” refers to a molecule that has an average of greater than 1.0 hydroxyl groups per molecule. It may also include other functionalities. Polyols derived from natural oils are well known in the art.

Examples of polyols that can be subjected to the method of the present invention include polyols prepared as described in U.S. Pat. No. 6,433,121 (Petrovic et al.). These polyols can be made by ring opening epoxidized soybean oil with a mixture of methanol and water) which provides a high hydroxyl value, such as in the range of about 200-230 mg KOH/g.

Polyols can also be made by ring opening epoxidized soybean oil with only methanol, which provides a lower hydroxyl value, such as in the range of about 160-190 mg KOH/g, or a hydroxyl value.

In some cases, the polyol of a natural oil-based product is an oligomeric polyol. As used herein “oligomeric polyol” refers to a non naturally occurring polyol prepared by rings opening a fully or partially epoxidized natural oil (such as a plant-based oil or an animal fat) in a manner that results in the formation of oligomers. The oligomeric polyol can be an oligomer of two or more triglyceride-based monomers that have been chemically bonded to one another by ether linkages during an epoxide ring-opening reaction. Oligomers include dimers, trimers, tetramers, and higher order oligomers.

Examples of patents describing oligomeric polyols include International Publication Nos. WO06/116456; WO05/033167; and WO06/012344. Other examples of oligomeric polyols that can be subjected to the method of the present invention includes polyols prepared as described in U.S. Patent Application No. 60/795,327, filed Apr. 27, 2006, and entitled “Enhanced Oligomeric Polyols” (Abraham et al.) published as International Publication No. WO07/127,379A1.

The natural oil-based product can include a mixture of oligomerized polyols, having different molecular weights, and can also include some non-oligomerized polyols. More specifically, the natural oil can be a plant oil, such as soybean oil or a palm-based oil. An example of an oligomeric polyols is described in International Publication No. WO06/012344A1 (Petrovic et al.), some of the details of which are described herein.

The preparation of polyols and oligomeric polyols is generally, and more specifically, described herein. In some particular aspects of the invention, the polyols and oligomeric polyols can be prepared as described herein and then subjected to the deodorization process of the present invention.

The natural oil-based product subjected to deodorization according to the present invention can include polyols and oligomeric polyols that are prepared by ring-opening an epoxidized natural oil. In many embodiments, the ring-opening is conducted using a reaction mixture comprising: (1) an epoxidized natural oil, (2) a ring-opening acid catalyst, and (3) a ring-opener. These materials are described in more detail herein. The natural oil-based product can also include modified vegetable oil-based polyols such as described in WO06/012344A1 (Petrovic et al.).

Epoxidized natural oils include, for example, epoxidized plant-based oils (e.g., epoxidized vegetable oils) and epoxidized animal fats. The epoxidized natural oils may be partially or fully epoxidized. Partially epoxidized natural oil may include at least about 10% or more of the original amount of carbon-carbon double bonds present in the oil. The partially epoxidized natural oil may include up to about 90%, or fewer, of the original amount of carbon-carbon double bonds present in the oil. Fully epoxidized natural oil may include up to about 10%, or fewer, of the original amount of carbon-carbon double bonds present in the oil.

Examples of natural oils include plant-based oils (e.g., vegetable oils) and animal fats. Examples of plant-based oils include soybean oil, safflower oil, linseed oil, corn oil, sunflower oil, olive oil, canola oil, sesame oil, cottonseed oil, palm-based oils, rapeseed oil, tung oil, peanut oil, and combinations thereof. Animal fats may also be used, for example, fish oil, lard, and tallow. The plant-based oils may be natural or genetically modified vegetable oils, for example, high oleic safflower oil, high oleic soybean oil, high oleic peanut oil, high oleic sunflower oil, and high erucic rapeseed oil (crambe oil).

The number of carbon-carbon double bonds per molecule in a natural oil can be quantified by the iodine value (IV) of the oil. Typically, iodine values for the vegetable oils will range from about 40 to about 240. In some embodiments, vegetable oils having an iodine value greater than about 80 are used. In some embodiments, vegetable oils having an iodine value less than about 240 are used.

Useful natural oils comprise triglycerides of fatty acids. The fatty acids may be saturated or unsaturated and may contain chain lengths ranging from about C12 to about C24. Unsaturated fatty acids include monounsaturated and polyunsaturated fatty acids. Common saturated fatty acids include lauric acid (dodecanoic acid), myristic acid (tetradecanoic acid), palmitic acid (hexadecanoic acid), stearic acid (octadecanoic acid), arachidic acid (eicosanoic acid), and lignoceric acid (tetracosanoic acid). Common monounsaturated fatty acids include palmitoleic (a C16 unsaturated acid) and oleic (a C18 unsaturated acid). Common polyunsaturated fatty acids include linoleic acid (a C18 di-unsaturated acid), linolenic acid (a C18 tri-unsaturated acid), and arachidonic acid (a C20 tetra-unsaturated acid).

The triglyceride oils are made up of esters of fatty acids in random placement onto the three sites of the trifunctional glycerine molecule. Different vegetable oils will have different ratios of these fatty acids.

In an exemplary embodiment, the natural oil-based product subjected to the process of the present invention is prepared from a fully epoxidized soybean oil. Although not wishing to be bound by theory, it is believed that the use of saturated epoxidized vegetable oils (that is, having little or no residual carbon-carbon double bond functionality) having residual epoxy groups leads to oligomeric polyols having good oxidative stability.

In another exemplary embodiment, the natural oil-based product subjected to process of the present invention is prepared from a palm-based oil product. As used herein “palm-based oil” refers to an oil or oil fraction obtained from the mesocarp and/or kernel of the fruit of the oil palm tree. Palm-based oils include palm oil, palm olein, palm stearin, palm kernel oil, palm kernel olein, palm kernel stearin, and mixtures thereof. Palm oil is typically a semisolid at room temperature and comprises about 50% saturated fatty acids and about 50% unsaturated fatty acids. Palm oil typically comprises predominately fatty acid triglycerides, although monoglycerides and diglycerides may also be present in small amounts.

Palm olein refers to the liquid fraction that is obtained by fractionation of palm oil after crystallization at a controlled temperature. Relative to palm oil, palm olein has a higher content of unsaturated fatty acids, for example, C18:1 and C18:2 fatty acids, and has a higher iodine value.

Palm stearin refers to the solid fraction that is obtained by fractionation of palm oil after crystallization at a controlled temperature. Relative to palm oil, palm stearin contains more saturated fatty acids and has a higher melting point.

A partially epoxidized or fully epoxidized natural oil may be prepared by a method that comprises reacting a natural oil with a peroxyacid under conditions that convert a portion of or all of the carbon-carbon double bonds of the oil to epoxide groups. Examples of peroxyacids include peroxyformic acid, peroxyacetic acid, trifluoroperoxyacetic acid, benzyloxyperoxyformic acid, 3,5-dinitroperoxybenzoic acid, m-chloroperoxybenzoic acid, and combinations thereof. Additional acids such as sulfuric acid, para-toluenesulfonic acid, trifluoroacetic acid, fluoroboric acid, Lewis acids, acidic clays, or acidic ion exchange resins can be used in the reaction. Optionally solvents, such as aprotic solvents, can be used in the reaction. Representative examples of suitable solvents include benzene, toluene, xylene, hexane, isohexane, pentane, heptane, and chlorinated solvents (e.g., carbon tetrachloride).

Subsequent to the epoxidation reaction and prior to the deodorization process, the reaction product can be neutralized. A neutralizing agent may be added to neutralize any remaining acidic components in the reaction product. Suitable neutralizing agents include weak bases, metal bicarbonates, or ion-exchange resins. Examples of neutralizing agents that may be used include ammonia, calcium carbonate, sodium bicarbonate, magnesium carbonate, amines, and resin, as well as aqueous solutions of neutralizing agents. Typically, the neutralizing agent will be an anionic ion-exchange resin. If a solid neutralizing agent (e.g., ion-exchange resin) is used, the solid neutralizing agent may be removed from the epoxidized vegetable oil by filtration. Alternatively, the reaction mixture may be neutralized by passing the mixture through a neutralization bed containing a resin or other materials. Alternatively, the reaction product may be repeatedly washed to separate and remove the acidic components from the product. In addition, one or more of the processes may be combined in neutralizing the reaction product. For example, the product could be washed, neutralized with a resin material, and then filtered.

Useful fully-epoxidized soybean oils include, for example, but not limited to commercially available products such as those under the trade designations EPOXOL™ 7-4 (from American Chemical Systems), Drapex™ 6.8 (from Chemtura), and FLEXOL™ ESO (from Dow Chemical Co.).

The ring-opening reaction that leads to production of polyols or oligomeric polyols is conducted in the presence of a ring-opening acid catalyst. Representative examples of ring-opening acid catalysts include Lewis or Brönsted acids. Examples of Brönsted acids include hydrofluoroboric acid (HBF₄), triflic acid, sulfuric acid, hydrochloric acid, phosphoric acid, phosphorous acid, hypophosphorous acid, boronic acids, sulfonic acids (e.g., para-toluene sulfonic acid, methanesulfonic acid, and trifluoromethane sulfonic acid), and carboxylic acids (e.g., formic acid and acetic acid). Examples of Lewis acids include phosphorous trichloride and boron halides (e.g., boron trifluoride).

The ring-opening reaction that leads to production of preferred polyols or oligomeric polyols is also conducted in the presence of a ring-opener. Various ring-openers may be used including alcohols, water (including residual amounts of water), and other compounds having one or more nucleophilic groups. Combinations of ring-openers may be used. For example, the ring-opener can be a monohydric alcohol. Representative examples include methanol, ethanol, propanol (including n-propanol and isopropanol), and butanol (including n-butanol and isobutanol), and monoalkyl ethers of ethylene glycol (egg, methyl cellosolve, butyl cellosolve, and the like). For example, the ring opening alcohol can be methanol. In some cases, the ring-opener can be a polyol. Polyol ring-openers include, for example, ethylene glycol, propylene glycol, 1,3-propanediol, butylene-glycol, 1,4-butane diol, 1,5-pentanediol, 1,6-hexanediol, polyethylene glycol, and polypropylene glycol. Also useful are vegetable oil-based polyols.

Oligomerized polyols can be formed by conducting the ring-opening reaction with a ratio of ring-opener to epoxide that is less than stoichiometric. This promotes oligomerization of the resulting ring-opened polyol. For example, an oligomeric polyol is prepared by reacting fully epoxidized soybean oil (ESBO) with methanol in the presence of a ring-opening catalyst, for example, fluoroboric acid. Typically, the molar ratio of methanol to fully epoxidized soybean oil will range from about 0.3 to about 3.0, more typically ranging from about 0.3 to about 2.0. For example, the molar ratio of the methanol to the epoxidized soybean oil is about 0.33.

As another example, in the formation of non-oligomerized polyol, the molar ratio of the methanol to the epoxidized soybean oil is about 6.0.

Typically, at the start of the reaction, the fully epoxidized soybean oil has an epoxide oxygen content (EOC) ranging from about 6.8% to about 7.4%. The ring-opening reaction is preferably stopped before all of the epoxide rings are ring-opened. For some ring-opening catalyst, the activity of the catalyst decreases over time during the ring-opening reaction. Therefore, the ring-opening catalyst may be added to the reactive mixture at a controlled rate such that the reaction stops at (or near) the desired endpoint EOC. The ring-opening reaction may be monitored using known techniques, for example, hydroxyl number titration (ASTM E1899-02), EOC titration (AOCS Cd9-57 or ASTM D1652 methods) or monitoring the heat removed from the exothermic reaction.

Typically, when fully epoxidized soybean oil is used, the ring-opening reaction is stopped when the residual epoxy oxygen content (EOC) ranges from about 0.01% to about 6.0%, for example, about 0.5% to about 5.5%, about 1% to about 5.0%, about 2% to about 4.8%, about 3% to about 4.6%, or about 3.5% to about 4.5%. When other epoxidized natural oils are used, the residual epoxy oxygen content (EOC) of the polyol may be different. For example, for palm oil, the residual EOC may range from about 0.01% to about 3.5%, for example, about 0.2% to about 3.0%, about 0.5% to about 2.0%, or about 0.8% to about 1.5%. As used herein “epoxy oxygen content” or “EOC” refers to the weight of epoxide oxygen in a molecule expressed as percentage.

During the ring-opening reaction, some of the hydroxyl groups of the ring-opened polyol react with epoxide groups that are present on other molecules in the reactive mixture (e.g., molecules of unreacted fully epoxidized soybean oil or molecules of polyol having unreacted epoxide groups) resulting in oligomerization of the polyol (i.e., the formation of dimers, trimers, tetramers, and higher order oligomers). The degree of oligomerization contributes to the desired properties of the oligomeric polyol including, for example, number average hydroxyl functionality, viscosity, and the distance between reactive hydroxyl groups. As an example, the oligomeric polyol comprises about 40% weight or greater oligomers (including dimers, trimers, and higher order oligomers). As another example, the oligomeric polyol comprises about 35% to about 45% weight monomeric polyol and about 55% to about 65% weight oligomers (e.g., dimers, trimers, tetramers, and higher order oligomers). As another example, the oligomeric polyol comprises about 35% to about 45% weight monomeric polyol, about 8% to about 12% weight dimerized polyol, about 5% to about 10% weight trimerized polyol, and about 35% weight or greater of higher order oligomers.

For an exemplary non-oligomeric polyol, the monomer content is >70%, typically 75-82%.

Oligomerization may be controlled, for example, by catalyst concentration, reactant stoichiometry, and degree of agitation during ring-opening. Oligomerization tends to occur to a greater extent, for example, with higher concentrations of catalyst or with lower concentration of ring-opener (e.g., methanol). Upon completion of the ring-opening reaction, any unreacted methanol is typically removed, for example, by vacuum distillation. Unreacted methanol is not desirable in the oligomeric polyol because it is a monofunctional species that will end-cap the polyisocyanate. After removing any excess methanol, the resulting polyol is typically filtered, for example, using a 150 micron bag filter in order to remove any solid impurities.

The oligomeric polyols produced can have a low number average hydroxyl functionality. Number average hydroxyl functionality refers to the average number of pendant hydroxyl groups (e.g., primary, secondary, or tertiary hydroxyl groups) that are present on a molecule of the polyol. For example, the oligomeric polyol has a number average hydroxyl functionality (Fn) about 2.7 or less, for example, about 2.6 or less, about 2.5 or less, about 2.4 or less, about 2.3 or less, about 2.2 or less, about 2.1 or less, about 2.0 or less, about 1.9 or less, about 1.8 or less, about 1.7 or less, about 1.6 or less, about 1.5 or less, or about 1.4 or less. Typically, the number average hydroxyl functionality ranges from about 1.5 to about 2.4 or from about 1.7 to about 2.2.

In some cases, the oligomeric polyol has a hydroxyl number (OH number) that ranges from about 45 to about 65 mg KOH/g, or from about 55 to about 65 mg KOH/g.

Hydroxyl number indicates the number of reactive hydroxyl groups present on the oligomeric polyol. It is expressed as the number of milligrams of potassium hydroxide equivalent to the hydroxyl content of one gram of the sample.

In some cases, the oligomeric polyol has a low acid value. Acid value is equal to the number of milligrams of potassium hydroxide (KOH) that is required to neutralize the acid that is present in one gram of a sample of the polyol (i.e., mg KOH/gram).

In some cases, the oligomeric polyol has an acid value that is less than about 5 (mg KOH/gram), for example, less than about 4 (mg KOH/gram), less than about 3 (mg KOH/gram), less than about 2 (mg KOH/gram), or less than about 1 (mg KOH/gram). As another example, the acid value is less than about 1 (mg KOH/gram), for example, less than about 0.5 (mg KOH/gram), or from about 0.2 to about 0.5 (mg KOH/gram).

In some cases, the number average molecular weight (i.e, Mn) of the oligomeric polyol is about 1000 grams/mole or greater, for example, about 1100 grams/mole or greater, about 1200 grams/mole or greater, about 1300 grams/mole or greater, about 1400 grams/mole or greater, or about 1500 grams/mole or greater. In some cases, the Mn is less than about 5000 grams/mole, for example, less than about 4000 grams/mole, less than about 3000 grams/mole, or less than about 2000 grams/mole. In some cases, the Mn ranges from about 1000-5000 grams/mole, for example, about 1200-3000 grams/mole, about 1300-2000 grams/mole, about 1700-1900 grams/mole, or about 1500-1800 grams/mole. Number average molecular weight may be measured, for example, using light scattering, vapor pressure osmometry, end-group titration, and colligative properties.

In some cases, the weight average molecular weight (i.e, Mw) of the oligomeric polyol is about 5000 grams/mole or greater, for example, about 6000 grams/mole or greater, about 7000 grams/mole or greater, or about 8000 grams/mole or greater. In some cases, the Mw is less than about 50000 grams/mole, for example, less than about 40,000 grams/mole, less than about 30000 grams/mole, or less than about 20000 grams/mole. In some embodiments, the Mw ranges from about 5000-50000 grams/mole, for example, about 5000-20000 grams/mole, or about 6000-15000 grams/mole. Weight average molecular weight may be measured, for example, using light scattering, small angle neutron scattering (SANS), X-ray scattering, and sedimentation velocity.

Typically the oligomeric polyol has a polydispersity (Mw/Mn) of about 3-15, for example, about 4-12, or about 5-10.

Generally, an oligomeric polyol is produced having a viscosity of about 500 cP or greater. In some embodiments, the oligomeric polyol has a viscosity at 25° C. of about 500 to about 10000 cP (about 0.5 to about 10 Pa·s). When soybean oil is used as a starting material, the viscosity of the oligomeric polyol typically ranges from about 2000 cP to about 8000 cP, or from about 3000 cP to about 7000 cP (2 to about 8 Pa·s, or from about 3 to about 7 Pa·s).

When a palm-based oil is used, the viscosity of the oligomeric polyol is typically in the range of about 0.5 Pa·s to about 2 Pa·s.

In some cases, the oligomeric polyol has few, if any, residual carbon-carbon double bonds. This is particularly true if the oligomeric polyol is prepared from fully epoxidized soybean oil. One measure of the amount of carbon-carbon double bonds in a substance is its iodine value (IV). The iodine value for a compound is the amount of iodine that reacts with a sample of a substance, expressed in centigrams iodine (I₂) per gram of substance (cg I₂/gram). In some cases, the oligomeric polyol has an iodine value that is less than about 50, for example, less than about 40, less than about 30, less than about 20, less than about 10, or less than about 5.

For non-oligomeric polyols, the viscosity can be higher due the presence of unreacted hydroxyl groups. For example, the polyol can have a viscosity in the range of about 8000 cP to about 11000 cP at 25° C. As another example, the polyol can have a viscosity in the range of about 4000 cP to about 6000 cP at 25° C.

The process of the present invention can be used to reduce the amount of volatilizable components in a natural oil-based product. Volatilizable components include both components that can impart distinctive odors to a product, as well as components that do impart distinctive odors.

In some aspects, the steps of the present invention are carried out in order to remove odor components from a natural oil-based product. In the process, other volatilizable components may be removed from the natural oil-based product. The removal of other components can provide additional benefits to a deodorized natural oil-based product.

The method of the present invention can provide a natural oil-based product (e.g., a natural oil-based polyol) having levels of odor components (as measured by the combined levels of hexanal, nonanal, and decanal) of about 25 ppm or less, about 20 ppm or less, about 15 ppm or less, more preferably about 10 ppm or less, about 5 ppm or less, about 4 ppm or less, about 3 ppm or less, and most preferably about 2 ppm or less, or about 1 ppm or less. The process of the present invention can be used to provide a “deodorized” natural oil-based product.

In more specific aspects, the invention provides a deodorized natural oil-based polyol having a viscosity of about 500 cP or greater, about 2000 cP or greater, or about 3000 cP or greater; a level of odor components (as measured by the combined levels of hexanal, nonanal, and decanal) of about 25 ppm or less, about 20 ppm or less, about 15 ppm or less, about 10 ppm or less, about 5 ppm or less, about 4 ppm or less, about 3 ppm or less, about 2 ppm or less, or about 1 ppm or less; and a Gardner color value of about 2.0 units or less, about 1.5 units or less, or of about 1.

According to one aspect of the invention, a natural oil-based product having an elevated viscosity is subjected to a deodorization process to remove odor components. In some preferred aspects, the natural oil-based product includes an oligomerized polyol, such as described herein, which is subjected to the deodorization process as described.

In accordance with the inventive methods described herein, deodorization can be performed using standard deodorization equipment. In this regard, the methods of the invention can be carried out in deodorization facilities traditionally used for the deodorization of common vegetable oils. The addition of modification of deodorization equipment in these facilities is not required, but optional.

In its most fundamental form, the following steps of the method are followed. First, the natural oil-based product having an elevated viscosity of about 500 cP or greater is first provided by a user and placed in a deodorization apparatus. In the deodorization apparatus the natural oil-based product is heated to a temperature of about 225° C., or less, for a period of time. After the oil-based product is heated for a desired period of time, it is exposed to another portion of the deodorization apparatus having an environment comprising a reduced pressure and a sparging vapor. The step of exposing is carried out for a period of time sufficient for removal of the majority of one or more volatilizable components that are associated with the natural oil-based product. In many cases the portion of the deodorization apparatus having an environment comprising a reduced pressure and a sparging vapor is a deodorization column, which is discussed in greater detail herein.

Heating the natural oil-based product to a very high temperature which would otherwise cause its discoloration, is not required. For example, the process of the invention is carried out wherein temperature of the natural oil-based product does not exceed 225° C., or does not exceed 220° C., during the steps of the process.

While these fundamental steps are carried out in the deodorization process, the process can include additional steps and equipment. These additional steps and equipment may be standard on traditional deodorization apparatus and methods, and therefore the methods of the invention may typically include them when the deodorization process is carried out. One or more additional steps can be performed before, between, or after the basic steps in the deodorization method described. Again, if additional steps are performed it is not required that the product be heated to a temperature that exceeds 225° C., or that exceeds 220° C.

As an example, the process can include a step of deaerating the natural oil-based product, which is performed before the step of heating. The method can also include sub-steps, which are more detailed steps performed within a step inventive method. Various combinations of additional steps and equipment can be used with along with the fundamental steps of the inventive method described herein. One of skill in the art would understand that techniques or equipment known in the art could be employed in the present inventive method.

The process of the present invention is suitable for industrial scale production of a deodorized oil-based product, as well as for pilot scale productions. As an example, a scaled up industrial processes is capable of producing amounts of deodorized oil-based product in excess of 500 kg, and more typically in excess of 20,000 kg.

The method of the invention can be carried out using a batch, semi-continuous, or continuous process. In preferred aspects of the invention, the natural oil-based product is treated in a continuous or semi-continuous process. For example, in a semi-continuous process, the natural oil-based product is flowed through the apparatus, and at points in the method (such as the heating step) the flow can be hindered, and a bulk portion of the natural oil-based product can be treated.

The method of the invention is herein exemplified by the deodorization of a natural oil-based product in a semi-continuous process using a deodorization apparatus that is commonly used for the deodorization of vegetable oils. Prior to introduction into the deodorization apparatus, the natural oil-based product can be preheated. This can reduce the amount of time needed to heat the natural oil-based product to elevated temperatures in the apparatus. For example, the natural oil-based product can be pre-heated before being introduced into the apparatus.

The natural oil-based product can be drawn into a feed tank of the apparatus and “preconditioned” prior to subjecting the product to elevated temperatures for removal of the volatilizable components. In the feed tank heat can be applied to further heat the natural oils based product. For example, in one mode of practice, the product is heated to a temperature of about 100° C. The oil-based product can also be agitated while in the feed tank to promote its uniform heating. In the feed tank the product can be contacted with an inert gas, such as nitrogen, to prevent unwanted oxidation of the product prior to the deodorization process. Oxygen can accordingly be purged from the feed tank.

The oil-based product can be maintained in the feed tank for a desired period of time. Once the oil-based product has reached a desired condition (e.g., temperature), it can be provided to a subsequent vessel that is in liquid communication with the feed tank.

In some modes of practice, the oil-based product is subjected to a step of deaeration. Deaeration can be performed prior to subjecting the product to elevated temperatures and removal of the volatilizable components. The decision on whether to perform a deaeration step can depend one or more factors, such as the level of dissolved gasses in the natural oil-based product. A deaeration step can remove gasses dissolved in the natural oil-based product, such as oxygen, to facilitate downstream steps when a strong vacuum is applied to the natural oil-based product. Additionally, deaeration can reduce the amount of water and methanol, as well as other very low boiling point volatiles that may be present in the natural oil-based product.

Deaeration can be performed by drawing the natural oil-based product into a deaeration vessel, and while the oil-based product is kept at an elevated temperature (e.g., of about 100° C.), subjecting the product to a moderate vacuum. Removal of dissolved gas from the oil-based product can improve downstream processes such as deodorization, wherein a high vacuum is typically used. Pressure in the deaeration vessel is generally maintained at about 25 in Hg (Torr) or less. In some modes of practice the residence time of the oil-based product in the deaeration vessel is in the range of about 15 to about 20 minutes, although longer times may be used.

Mixing of the natural oil-based product in the deaeration vessel can be performed. For example, in some cases, after the step of deaerating is performed, and prior to heating the product, the natural oil based product can be pumped from the deaeration vessel and back into the deaeration vessel via a recirculation conduit. Recirculation to the deaeration vessel can be controlled by appropriate valving. In some modes of practice, the natural oil-based product is recirculated back to the deaeration vessel at a rate of about 0.5 gallons per minute (GPM).

If a step of deaeration is performed, it is generally followed by the step of heating the natural oil-based product. Heating is performed to increase the temperature of the natural oil-based product to a temperature of about 225° C. or below. In some preferred modes of practice the natural oil-based product is heated to a temperature in the range of about 215° C. to about 220° C., such as about 217° C. or 218° C.

Such heating can be accomplished by flowing the natural oil-based product through a heat exchanger. Within the heat exchanger, the natural oil-based product can be heated, and then discharged to a retention vessel when the desired temperature is reached. The heat exchanger can include input and solenoid discharge valves to control the flow of the natural oil-based product into and out of the heat exchanger. The solenoid discharge valve can be set at a desired temperature to control release of the natural oil-based product.

The heat exchanger can increase the temperature of the natural oil-based product by at least 100° C. For example, in some modes of practice, the natural oil-based product enters the heat exchanger at a temperature of about 100° C. and is discharged at a temperature in the range of about 215° C. to about 220° C.

One example of a suitable heat exchanger is a shell and tube heat exchanger. A shell and tube heat exchanger consists of a bundle of tubes enclosed in a cylindrical shell. The ends of the tubes are fitted into tube sheets, which separate the shell side and tube side fluids. Baffles are provided in the shell to direct the fluid flow and support the tubes. Support rods and spacers hold the assembly of baffles and tubes together.

The shell and tube heat exchangers can be constructed with a very large heat transfer surface in a relatively small volume. Exchangers can be fabricated from alloy steels to resist corrosion, and which are also useful for heating. The tubes are connected so that the internal fluid makes several passes up and down the exchanger thus enabling a high velocity of flow to be obtained for a given heat transfer area and throughput of fluid. The fluid flowing in the shell is made to flow first in one direction and then in the opposite across the tube.

The heating loop can include a thermal oil loop that runs at a temperature of about 30° C. warmer than the desired discharge temperature of the natural oil-based product. For example, in one mode of practice, the natural oil-based product is heated to a temperature of about 218° C. by a thermal oil loop that runs at a temperature of about 248° C.

The natural oil-based product can be discharged from the heat exchanger to a retention vessel at a desired flow rate. For example, in one mode of practice, the natural oil-based product is discharged from the heat exchanger at a rate of about 168 kg/hour.

The natural oil-based product is discharged at a desired temperature, which can be about 225° C. or below. Most preferably, the natural oil-based product is discharged at a temperature in the range of about 215° C. to about 220° C., such as about 218° C. However, the natural oil-based product can be discharged at a lower temperature, such as in the range of about 210° C. to about 220° C., about 205° C. to about 220° C., about 200° C. to about 220° C., about 190° C. to about 220° C., about 195° C. to about 220° C., or even about 185° C. to about 220° C.

After the natural oil-based product is heated to the desired temperature of about 225° C. or below, and prior to being subjected to deodorization, the natural oil-based product can be delivered to, and maintained in a retention vessel for a desired period of time at the elevated temperature.

In some modes of practice, the natural-oil-based product is maintained in the retention tank for a period of time in the range of about 8 to about 15 minutes at a temperature of about 225° C. or below. For example, in preferred modes of practice, the natural oil-based product is maintained in the retention tank at a temperature in the range of about 215° C. to about 220° C. for a period of time in the range of about 8 to about 15 minutes. If lower temperatures are used, longer retention times can be applied.

Treatment of the natural oil-based product at the elevated temperature results in a reduction in the peroxide value. Reduction in peroxide value is desirable as it is thought to prevent reappearance of odor components as a result of decomposition of peroxides during later process steps or during storage. Temperatures in the range of about 215° C. to about 220° C. were able to provide an excellent reduction in the peroxide value of the natural oil-based product. As an example, the peroxide value of the polyol material prior to deodorization was about 3.5, and following deodorization, the peroxide value decreased to about 1 (as measured by AOCS test Cd 8b-90). As another example, the peroxide value of the polyol material prior to deodorization was about 1.0, and following deodorization, the peroxide value decreased to about 0.2. Peroxide value can be measured by AOCS method Cd 8b-90 and reported in (meq peroxide/1000 grams sample).

Therefore, the natural oil-based product can be maintained at the elevated temperature and for a period of time to reduce the peroxide value in the product by at least 50%, by at least 60%, by at least 70%, by at least 80%, or even by at least 90%.

It was also discovered that these temperatures were not only useful for reducing peroxide value, but also for preventing a return to increased peroxide values after the natural oil-based product was subjected to the methods of the present invention. In particular, temperatures in the range of about 215° C. to about 220° C. sufficiently prevented a return to higher peroxide values when the deodorized product was protected from oxygen.

In addition, the process of the present invention prevented the formation of an undesirable polyol film on the walls of the deodorization equipment. (Films were seen at temperatures of greater than 225° C.)

In the retention vessel, a vacuum can be applied to the natural oil-based product to reduce pressure. The pressure can be reduced to levels near, or at, the level that is applied in the step of exposing the natural oil-based product to reduced pressure and sparging vapors. By the application of vacuum, any vapors that are generated in the retention tank can be removed via the vacuum system. In some preferred modes of practice, the retention vessel is placed in vacuum equilibrium with the deodorization vessel, so that when the natural oil-based product is transferred, abrupt changes pressure are avoided.

For example, vacuum can be applied in the retention vessel to reduce the pressure to about 5 Torr or below, or about 3 Torr or below. Therefore, within the step of heating, a sub-step of subjecting the natural oil-based product to a reduced pressure environment can be performed.

Following the step of heating, the process includes a step of exposing the natural oil-based product to an environment comprising a reduced pressure and a sparging vapor. The deodorization is typically carried out under high vacuum. Pressure in the deodorization column is generally maintained at about 5 Torr or less, and preferably about 3 Torr or less. The sparging vapor is any gas that is compatible with the natural oil-based product, such as steam or nitrogen.

The step of exposing is carried out for a period of time sufficient for removal of the majority of one or more volatilizable components that are associated with the natural oil-based product. The step of exposing can involve removal of volatilizable components that are odor components, from the natural oil-based product.

In a preferred method of the invention, the natural oil-based product is deodorized in a deodorization column under reduced pressure and in the presence of a sparging vapor. In particular preferred modes of practice the natural oil-based product is deodorized in a continuous countercurrent sparging vapor column. The column typically includes a packing material over which the natural oil-based product flows. For example, in a countercurrent column the sparging vapor and natural oil-based product travel in opposite directions. In typical countercurrent columns, the product subject to treatment (i.e., the natural oil-based product) is fed into the top of vertically oriented column and then travels downwards through the column by gravity. Sparging vapor is introduced in the column, generally at the bottom of the column, but can be introduced at multiple vertical locations in the column. The sparging vapor travels upward as pulled by the vacuum system as the natural oil-based product travels downward through the column.

Various columns and column materials are known the art and useful for the deodorization process described herein.

Any one or a combination of various packing materials present in the column, or a structural arrangement in the column, can increase the surface area of the natural oil-based product that contacts the sparging vapor. One or more various types of packing materials can be present in the column. These include thin film packing materials formed of inert and thermoresistant materials, such as metals, metal alloys, thermoplastics, and ceramic materials.

The column can have dimensions, and include particular length to diameter ratios [L:D ratio] to reflect the surface area of the packing material on which the natural oil-based product spreads over as it travels downward in the column. In one mode of practice, the L:D ratio is about 22.4:1.

Contact with the sparging vapors will generally cause a decrease in the temperature of the natural oil-based product. The temperature decrease may begin as the natural oil-based product travels from the retention vessel into the top of the deodorization column. Cooling of the natural oil-based product continues as it spreads over the packing materials and travels downward in the column, which results in a dramatic increase in the surface area of the natural oil-based product and contact with the sparging vapors. Some loss of heat can also occur from the wall of the column, which can be optionally insulated to prevent this loss. However, the increase of surface area in combination with the high vacuum and sparging vapor drives the odor components and other low boiling point components out of the natural oil-based product and into the sparging vapor countercurrent.

A sparging vapor is provided to the column which improves removal of the low boiling point odor components from the natural oil-based product. Any suitable sparging vapor can be used. For example, in a preferred mode of practice, water vapor (steam) is used as the sparging vapor. Nitrogen can also be used as the sparging vapor.

Sparging vapor can be introduced at one or more locations (i.e. a sparger inlet) in the equipment, and/or at one or more locations in the column and cooling reservoir. In one preferred mode of practice, the sparging vapor is provided to the column or cooling reservoir.

The one or more sparger inlets can be in gaseous communication with a manifold, which provides the sparging vapors. In one mode of practice, the sparging vapor is introduced into the column from the bottom of the column. For example, the sparging vapor can be introduced into the cooling reservoir at the bottom of the column. The sparger inlet can be located so that it is submerged in the natural oil-based product present in the cooling reservoir. In this preferred method, a desirable distribution of sparging vapor through the natural oil-based product can be achieved before being pulled through the column.

In a preferred aspect of the present invention, the sparging vapors flow countercurrently to the natural oil-based product as the vapors are being pulled upward toward the vacuum system. In this step, volatile odor components are being pulled to the vacuum system as well.

The amount of sparging vapor delivered during the process can be measured as a percentage of the amount of sparging vapor delivered per amount of natural oil-based product processed (e.g., lbs steam/lbs polyol; w/w). In some aspects the amount of sparging vapor delivered during the step of deodorization is about 0.5% w/w or greater, about 0.75% w/w or greater, about 1.0% w/w or greater, about 1.25% w/w or greater, or about 1.5% w/w or greater.

During the step of deodorization the majority of the amount of one or more odor components is removed from the natural oil-based product. In many aspects the one or more odor components can be an aliphatic aldehyde(s). In particular, the one or more odor components can be selected from C₆-C₁₄ aliphatic aldehydes. These can be, but are not limited to, hexanal, nonanal, and decanal.

For example, one or more odor components in an amount of 75% or greater, 85% or greater, 90% or greater, or 95% or greater can be removed from natural oil-based product. As an example, for a natural oil based polyol product having a total level of odor components (as measured by the combined levels of hexanal, nonanal, and decanal) of about 40 ppm or less, a 95% (or greater) reduction in the odor components according to the process of the present invention, results in a deodorized polyol product having level of odor components (as measured by the combined levels of hexanal, nonanal, and decanal) of about 2 ppm or less.

After the natural oil-based product has finished flowing over the column, it accumulates at the bottom of the column where it is subjected to further cooling. The vapors of the sparging system also contribute to the cooling of the natural oil-based product. In one mode of practice, the temperature of the natural oil-based product in this vessel is about 125° C. In one mode of practice, the flowrate of the polyol leaving the column is about 189 kg/hr.

After the natural oil-based product has been subjected to column treatment to remove the volatilizable components, it can be further processed, if desired. For example, the deodorized natural oil-based product can be filtered to remove any particulates that are carried over from the apparatus or that form during the processing.

After processing, the deodorized natural oil-based product can be stored under desired conditions. For example, the deodorized product can be kept under a nitrogen or steam blanket to prevent contact with oxygen. The deodorized product can also be heated and agitated.

The present invention also provides a deodorized a natural oil-based product having reduced levels of volatilizable components. More particularly, the invention provides a deodorized natural oil-based product. The natural oil-based product has a viscosity of 500 cP or greater at 25° C.

Generally, the deodorized natural oil-based product (such as a deodorized polyol) has a combined hexanal, nonanal, and decanal content of about 25 ppm or less, 20 ppm or less, or 15 ppm or less. In some specific, preferred aspects the deodorized natural oil-based product has a combined hexanal, nonanal, and decanal content of about 10 ppm or less. In some specific aspects, the combined hexanal, nonanal, and decanal content is about 5 ppm or less, about 4 ppm or less, about 3 ppm or less, about 2 ppm or less, or about 1 ppm or less. In addition to the low levels of combined hexanal, nonanal, and decanal, the deodorized natural oil-based polyol can have a color on the Gardner scale of about 2.0 or less, of about 1.5 or less, or about 1.0 or less.

Odor may be measured, for example, by using human test panels or by measuring the amount of certain odor-producing compounds using analytical test methods.

The deodorized natural oil-based product, such as a deodorized polyol, can be used for the preparation of any one of a number of consumer products. The significant reduction in odor components is desirable in many aspects, as a product prepared from the deodorized natural oil-based product will be devoid of an unpleasant smell, and therefore acceptable to a consumer.

As an example, the deodorized natural oil-based product can be used in a method for the preparation of a polyurethane foam. In some embodiments the polyurethane foams of the invention exhibit little or no detectable odor, and a particularly suitable for commercial foam production.

### EXAMPLES

### I. Deodorization of High Viscosity Soybean Oil-Based Oligomeric Polyol

Oligomerized polymeric material was prepared according to WO06/012344A1 and deodorized in a commercial deodorization apparatus as follows.

Before deodorization, the polyol had a hydroxyl value of 57.63 (mg KOH/g), a oligomer concentration, wt % of 58.36, a viscosity of 3.89 Pa s⁻¹, a peroxide value of 2.47, an acid value of 0.34 (mg KOH/g), a Gardner color of less than 1, and a total odor content (combined hexanal, nonanal, and decanal) of 26 ppm.

Before feeding polyol to the deodorization apparatus, the apparatus was preheated and vacuum was applied. The scrubber recirculation pump was started to maintain a recirculation rate of 1200 LPM. Canola oil was used to preheat the deodorizer by introduction through the deaerator. The canola oil was then purged from the deodorizer.

The oligomeric polyol was pumped to the deaeration vessel and into the deodorizer at 168 kg/hr. 75.5 kg of polyol feed was used to help purge the canola oil before collecting the deodorized product.

The oligomeric polyol was pulled into the feed tank under vacuum and a nitrogen blanket was applied to the feed tank to prevent oxidation of the polyol. The tank was agitated and heated with jacketed steam to approximately 100° C.

From the feed tank, the polyol was pulled through a steam heater and then into the deaeration vessel (under ˜−25 in Hg). The steam heater heated the polyol up to 100° C. The temperature of the polyol in the deaeration vessel was 97° C. An isolation ball valve was used to control the level in the deaeration vessel. The level in the deaeration vessel was 40%.

The polyol was pumped from the deaeration vessel, with a portion being recycled back to the top of the deaeration vessel, which agitated the tank and facilitated deaeration. The remaining portion was pumped to the thermal oil heater.

The feed rate of the polyol to the deodorizer was 168 kg/hr. The polyol was heated in a shell and tube heat exchanger to a temperature of 217.6° C. and then discharged to a retention tank. The retention tank had one valve out of the three open to achieve a residence time of 14.5 minutes at 217.6° C. The retention tank was also in vacuum equilibrium with the deodorization column, which was a vacuum level of 3.0 torr. From the retention tank, the polyol flowed through piping before being feed into the top of the deodorization column. The temperature of the polyol at the top of the column was 210° C.

The polyol was flowed over Sulzer thin film structured Mellapak™ Plus 252Y 316L SS column material. The L/D ratio of the packing column is (216 mm″×16):154 mm=22.4:1. The countercurrent steam was applied at 2.5 kg/hr (1.5% wt % of the feed). The flowrate of the polyol leaving the column was 189 kg/hr.

As the polyol finished its flow over the packing, it accumulated at the bottom of the column where in a cooling water-jacketed 60 L reservoir vessel. The level in the vessel was maintained around 30 L at a temperature of 125° C. The polyol was then flowed through a set of two parallel canister filters having 20-micron filter socks and into a product tank where it was agitated, and heated above 60° C. using the steam jacket.

### II. Deodorization of High Viscosity Soybean Oil-Based Oligomeric Polyol

The process described in Example I shows one processing condition. A number of subsequent runs were performed with changes to following processing factors: (Factor 1) feed temperature, (Factor 2) retention time, and (Factor 3) sparge rate, as shown in Table 1 Variations in pressure were also tested. Odor content and product quality was determined following the runs.

TABLE 1

| | Factor 1 | Factor 2 | Factor 3 |
|---|---|---|---|
| | A: Temperature | B: Residence time | C: Steam sparging |
| Run | (deg C.) | (mins) | (kg/hr) |
| 1 | 260 | 8 | 2.52 |
| 2 | 175 | 14.25 | 2.52 |
| 3 | 217.5 | 14.75 | 2.52 |
| 4 | 260 | 21.5 | 4.2 |
| 5 | 260 | 21.5 | 0.84 |
| 5a | 260 | bypass holding | 0.84 |
| | | tank - 1 min | |
| 6 | 260 | 8 | 4.2 |
| 7 | 260 | 8 | 0.84 |
| 8 | 217.5 | 14.75 | 2.52 |
| 9 | 175 | 8 | 4.2 |
| 10 | 175 | 21.5 | 4.2 |
| 11 | 175 | 8 | 0.84 |
| 12 | 175 | 21.5 | 0.84 |
| 13 | 217.5 | 14.75 | 2.52 |
| 14 | 260 | 14.75 | 2.52 |
| 15 | 217.5 | 14.75 | 2.52 |
| 15a | 217.5 | bypass holding | 0.84 |
| | | tank - 1 min | |
| 16 | 217.5 | 8 | 2.52 |
| 17 | 217.5 | 8 | no sparging |
| 18 | 217.5 | 8 | no sparging, add N2 to |
| | | | vacuum to 5-10 torr at top |
| 19 | 217.5 | 8 | no sparging, add N2 to |
| | | | vacuum to 10-15 torr at top |
| 20 | 217.5 | 8 | add N2 to sparge until top |
| | | | vacuum is 3 torr |
| 21 | 175 | 8 | no sparging |
| 22 | 175 | 14.75 | 2.52 |
| 23 | 217.5 | 14.75 | 2.52 |
| 24 | 217.5 | 14.75 | 4.2 |
| 26 | 217.5 | 21.5 | 2.52 |

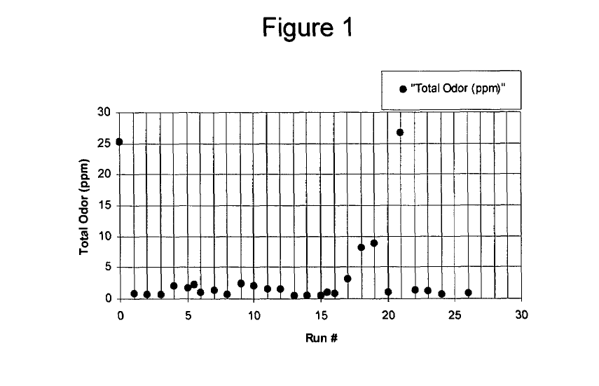

Total odor content of the polyol samples subjected to processing conditions from runs 1-26 was determined. Total odor content refers to the combined amount of hexanal, nonanal, and decanal, measured in parts per million (ppm) in the processed polyol samples as determined by gas chromatography (GC), FIG. 1 shows total odor content in the processed polyol samples from runs 1-26. (Run 0 is the total odor content in an unprocessed polyol sample.)

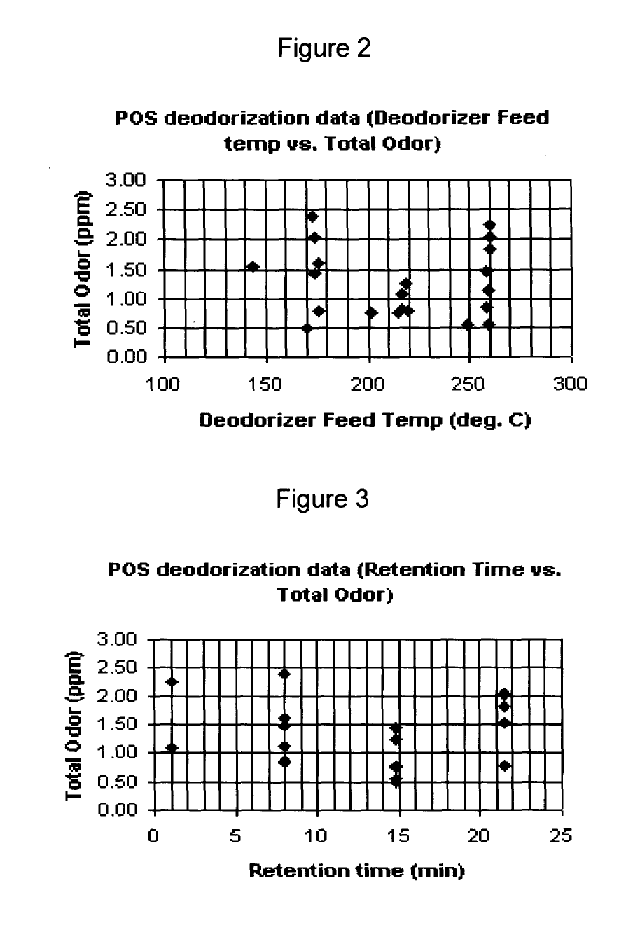

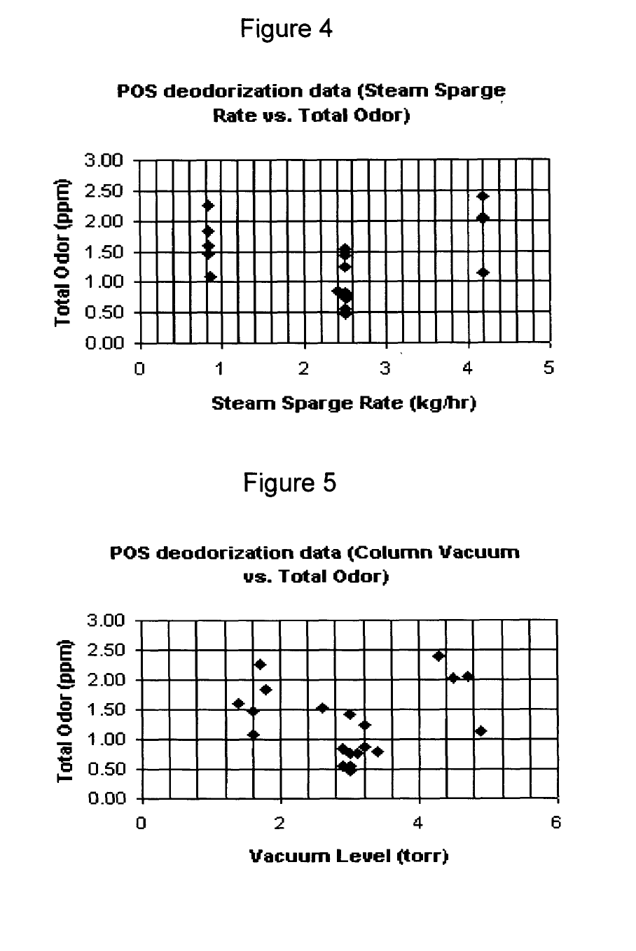

The data was also examined to determine what affect each factor (polyol feed temperature (FIG. 2), retention time (FIG. 3), steam sparge rate (FIG. 4), and column vacuum (FIG. 5)) has on total odor ppm level, the dependence of total odor from each condition on each factor was plotted. The data from runs 0, 18, 19, and 21 was excluded to prevent skewing of the plots.

### III. Quality Analysis of Deodorized Oligomeric Polyol Product

The oligomeric polyol product was subjected to analysis following the deodorization steps to verify that deodorization did not unacceptably alter the chemical and physical features of the polyol product. Standard polyol analysis was completed on the polyol at each condition and compared to the original starting polyol before deodorization. Table 2 below indicates the collective analytical data in terms of averages and comparing them the starting material results.

TABLE 2

| | | | | PV | AV | |
|---|---|---|---|---|---|---|
| | OH # (mg | GPC Total | Viscosity | (perox. | (acid | Color |
| Starting polyol | KOH/g) | Olig % | (Pa · s.) | value) | value) | (Gardner) |
| Average of all DO | 56.06 | 58.54 | 4.06 | 1.63 | 0.33 | 1.00 |
| conditions = | | | | | | |
| Max of all DO | 57.48 | 58.85 | 4.22 | 5.22 | 0.36 | <2 |
| conditions = | | | | | | |
| Min of all DO | 54.35 | 57.86 | 3.90 | 0.33 | 0.28 | <1 |
| conditions = | | | | | | |
| 260° C., 14.25 min. | 54.35 | 58.48 | 3.90 | 1.39 | .29 | 1.0 |
| res., 1.5% steam flow | | | | | | |
| 217.5° C., 14.25 min. | 56.71 | 58.74 | 4.10 | 1.05 | .33 | <1 |
| res., 1.5% steam flow | | | | | | |
| % change from | −2.7% | 0.3% | 4.5% | −34.0% | −4.0% | increase |
| original to average = | | | | | | |
| Specification of | 55-59 | | 2.8-4.2 | <3.5 | <0.5 | 1.0 or less |
| analytical method = | | | | | | |

Analysis of the deodorized product indicated that the 217.5° C. processing conditions resulted in minimal change in the polyol properties, as compared to the average of all deodorization conditions and the higher temperature (260° C.) processing conditions. The 260° C. processing conditions resulted in an undesirable color change in the polyol product, which did not occur at the 217.50° C. processing conditions.

Table 3 list the tests performed for product analysis.

TABLE 3

| Measurement | Test Method |
|---|---|
| Hydroxyl Number, mg KOH/g | AOCS Cd 13-60 or ASTM |
| | E1899 |
| Viscosity, cP @ 25 C. | DV-II+ Viscometer |
| Water, ppm | Coulometric Karl-Fischer |
| | ASTM E1064 |
| Acid Value, mg KOH/g | AOCS Cd 3d-63 |
| Pensky-Martens Flash, ° C. | ASTM D93 |
| Gardner Color | ASTM D1544 |
| Appearance | Visual inspection |
| Change in Epoxide Oxygen | AOCS Cd 9-57 or ASTM |
| Content, % | D1652 |
| Peroxide Value | AOCS Cd 8b-90 |
| Oligomer concentration, wt % | Gel permeation |
| | chromatography* |

1. A process for removing volatilizable components from a natural oil-based product comprising steps of:
(a) providing a natural oil-based product having a viscosity of 500 cP or greater at 25° C.;
(b) heating the natural oil-based product to a temperature of about 225° C. or below; and
(c) exposing the natural oil-based product to an environment comprising a reduced pressure and a sparging vapor, wherein volatilizable components are removed from the natural oil-based product.

2-5. (canceled)

6. The process of claim 1 where, in step (c), 50% or greater of the volatilizable components are removed from the natural oil-based product.

7-8. (canceled)

9. The process of claim 1 where the volatilizable components are selected from the group consisting of hexanal, nonanal, and decanal.

10. The process of claim 9 where, in step (a), the natural oil-based product comprises hexanal, nonanal, and decanal in a combined amount of 20 ppm or greater.

11. The process of claim 10 where, in step (c), hexanal, nonanal, and decanal are removed from the natural oil-based product to a combined amount of 10 ppm or less.

12. The process of claim 11 where, in step (c), hexanal, nonanal, and decanal are removed from the natural oil-based product to a combined amount of 5 ppm or less.

13. (canceled)

14. The process of claim 1 where, in step (b), the natural oil-based product is heated to a temperature in the range of 185° C. to 220° C.

15. The process of claim 14 where, in step (b), the natural oil-based product is heated to a temperature in the range of 205° C. to 220° C.

16. The process of claim 15 where, in step (b), the natural oil-based product is heated to a temperature in the range of 215° C. to 220° C.

17. The process of claim 1 where, in step (b), the natural oil-based product is heated for a period of time in the range of 8 to 15 minutes.

18. The process of claim 1 where the natural oil-based product has a peroxide value which is reduced by 50% or greater in step (b).

19. (canceled)

20. The process of claim 1 where, in step (c), the reduced pressure is 5 Torr or less.

21. The process of claim 1 where, in step (c), the sparging vapor is provided in an amount the range of 0.5% to 2.0%.

22. The process of claim 1 where step (c) is performed in a continuous countercurrent sparging vapor column.

23. The process of claim 1 wherein the natural oil-based product comprises a polyol.

24. The process of claim 23 where the natural oil-based product comprises an oligomeric polyol.

25. (canceled)

26. The process of claim 24 where the oligomeric polyol has a number average hydroxyl functionality less than about 2.5.

27. The process of claim 24 where the oligomeric polyol has an acid value that is less than about 1.0 mg KOH/gram.

28. The process of claim 24 where the oligomeric polyol has a number average molecular weight (Mn) of about 1000 to 5000 grams/mole.

29. (canceled)

30. The process of claim 24 where the oligomeric polyol has a residual epoxy oxygen content of about 0.01% to about 5.0%.

31. The process of claim 23 where the polyol is made from an epoxidized natural oil.

32-33. (canceled)

34. A process for deodorizing a natural oil-based product comprising steps of:
(a) providing a natural oil-based product having a viscosity of 500 cP or greater at 25° C.;
(b) heating the natural oil-based product to a temperature of about 225° C. or below; and (c) exposing the natural oil-based product to an environment comprising a reduced pressure and a sparging vapor, wherein hexanal, nonanal, and decanal are removed from the natural oil-based product.

35. A deodorized natural oil-based polyol comprising a viscosity of 500 cP or greater at 25° C., a combined hexanal, nonanal, and decanal content of 25 ppm or less and a Gardner color value of 2.0 or less.

36. The deodorized natural oil-based polyol of claim 35 where comprising a combined hexanal, nonanal, and decanal content of 5 ppm or less.

37. (canceled)

38. The deodorized natural oil-based polyol of claim 35 where comprising a Gardner color value of 1.5 or less.

39. A process for deodorizing a natural oil-based polyol comprising steps of:
(a) providing a natural oil-based polyol having a viscosity of 3000 cP or greater at 25° C.;
(b) heating the natural oil-based polyol to a temperature in the range of 185° C. to 220° C. wherein the temperature of the natural oil-based polyol in the process does not exceed 220° C.; and
(c) exposing the natural oil-based polyol to an environment comprising a reduced pressure and a sparging vapor, to provide a deodorized natural oil-based polyol having a combined hexanal, nonanal, and decanal content of 25 ppm or less and a Gardner color value of 2.0 or less.
